(19) United States
(12) Patent Application Publication
(10) Pub. No.: US 2010/0217389 A1
(43) Pub. Date: Aug. 26, 2010

(54) GRAFT ATTACHMENT DEVICE FOR LIGAMENT RECONSTRUCTION

(75) Inventors: Cheng-Kung CHENG, Taipei City (TW); Chih-Hwa CHEN, Taipei City (TW); Hung-Wen WEI, Taipei City (TW); Wen-Chuan CHEN, Taipei City (TW); Ping-Sheng YU, Taipei City (TW)

Correspondence Address: Muncy, Geissler, Olds & Lowe, PLLC, 4000 Legato Road, Suite 310, FAIRFAX, VA, 22033, US

(73) Assignee: NATIONAL YANG-MING UNIVERSITY, Taipei City (TW)

(21) Appl. No.: 12/467,878

(22) Filed: May 18, 2009

(30) Foreign Application Priority Data
Feb. 24, 2009 (TW) 098105868

(51) Int. Cl. A61F 2/08

(52) U.S. Cl. 623/13.14

(57) ABSTRACT

The present invention discloses a graft attachment device for cruciate ligament reconstruction comprises a body having a screw portion with a threading area on the front end of the body, a connection member disposed on the front end, a gap formed between the front end and a predetermined portion of the body adapted for a rope-type member pass through, a pivoting member pivoted on the rear end of the body with its front end, the rear end of the pivoting member having a juncture portion for fixing a ligament, thereby the device could be screwed and fixed on a bone when rotating the device, and seamed a ligament on the rear end of the device.

### BACKGROUND OF THE INVENTION

1. Field of the Invention

The present invention generally relates to medical equipment for ligament repair, and more particularly to a graft attachment device for cruciate ligament reconstruction.

2. Description of the Related Art

The method of ligament repair is positioning the patient's knee at an half-crooked angle, using a bore to bore and penetrate the femur and tibia simultaneously to form a 6 mm through hole, burying the ligament or insert member in the 6 mm through hole, and using any kinds of the graft attachment device to fix two ends of the ligament or insert member in the femur and tibia for replacing the damage cruciate ligament, so that the patient's bone and ligament or insert member are able to be healed gradually to recover the original functions of the knee with rehabilitation after surgery. However, the types of the known graft attachment device for fixing the ligament or insert member in the femur and tibia are of many different kinds, which the common seen is endo-button disclosed as U.S. Pat. No. 6,099,568 (ACL graft fixation device and method). The U.S. Pat. No. 6,099,568 is disclosed that the two ends of ligament or insert member wanted to be grafted are connected an endo-button respectively by using surgical suture. When the endo-button passes through in the pre-bored through hole of femur and tibia, the endo-button is using its side of smaller lateral cross-sectional area to pass through in the through hole, and then to turn over the endo-button after passing through out the openings of two ends of femur and tibia, so that the side of endo-button's largest cross-sectional area is locked at the openings of two ends of femur and tibia for fixing the ligament or insert member in femur and tibia. The endo-button is able to reduce wounds resulting from surgery, and by turning over the endo-button is able to fix the ligament or insert member in femur and tibia. But, two sides of the ligament or insert member connect the endo-button by elastic fiber cord or surgical suture. For having turn-over space after the endo-button passes through out the through hole, the endo-button must be pulled to tense the ligament or insert member to turn over, and then released, so that the endo-button elastically presses and closely combines at the opening of the through hole. The action mentioned as above could not make the ligament or insert member be tensed and fixed entirely in the bone, and rested a small depletion space at the part of the elastic fiber cord or surgical suture in the through hole connected with the ligament or insert member, so that the space could make the bone and ligament be healed more slowly. Moreover, when touching patient's skin after surgery, patient would have alien feeling.

There is another method for using interference screw to fix ligament or insert member disclosed as U.S. Pat. No. 6,589,245. This method is not able to rest more over depletion space compared to the endo-button mentioned as above, the surgical wound is small, and the patient is not able to have alien feeling after surgery. Furthermore, the interference screw is using inner fixing method, so as to make sure to pull tightly and position the ligament or insert member. However, since the method for fixing the ligament or insert member by interference screw is using the interference screw to oppress the ligament or insert member in the inner wall of the through hole of the bone and the portion of the ligament or insert member contacted the bone only has a surface, the ligament or insert member is made to be flat, so as to produce a gap after the interference screw is assimilated by human body and be not sure if the bone is able to grow into the gap and combine with the ligament or insert member entirely. And the structure and process of the tools for positioning and releasing the interference screw are complicated, so as to make the surgery process un-convenient, and the graft attachment device interfere the tools each other.

### SUMMARY OF THE INVENTION

It is an objective of the present invention to provide a graft attachment device for cruciate ligament reconstruction which has the advantages of convenient surgery operation and preventing the graft attachment device form twisting.

To achieve the above objectives, the present invention provides a graft attachment device for cruciate ligament reconstruction, comprising a body, having a screw portion disposed at an outer edge of the body and the screw portion having a threading area corresponding to a front end of the body; a connection member, fixed at the front end of the body, and a gap between the connection member and a corresponding portion of the body providing one rope-type member pass through; and a pivoting member, having a front end rotatably pivoted at a rear end of the body, and a connection portion disposed at a rear end of the pivoting member for installing a ligament thereon.

After the graft attachment device is inserted into the bones, operators can rotate the front end of the graft attachment device by an adaptive tool and the front end can be locked in the bone from inside to outside while the front end does not interfere the ligament connected at the rear end of the graft attachment device, so as to achieve the advantages of surgical wounds miniaturization and preventing the ligament form twisting and the tools from interfering with the graft attachment device while operating.

### BRIEF DESCRIPTION OF THE DRAWINGS

All the objects, advantages, and novel features of the invention will become more apparent from the following detailed descriptions when taken in conjunction with the accompanying drawings.

FIG. 1 shows a schematic diagram of an embodiment in accordance with the invention;

FIG. 2 shows a sectional drawing of the body of the embodiment in accordance with the invention;

FIG. 3 shows a sectional drawing of the graft attachment device of the embodiment in accordance with the invention;

FIG. 4 shows an isometric drawing of the connecting rod of the embodiment in accordance with the invention;

FIG. 5 shows an enlarged drawing of the distal end of the connecting rod of the embodiment in accordance with the invention;

FIG. 6 shows an enlarged drawing of the clamped portion of the connecting rod of the embodiment in accordance with the invention;

FIG. 7 shows a sectional drawing of the handle of the embodiment in accordance with the invention; and

FIG. 8 shows a schematic diagram of the embodiment when operating in accordance with the invention.

### DETAILED DESCRIPTION OF THE PREFERRED EMBODIMENT

Please refer to corresponding drawings. The invention is disclosed a graft attachment device for cruciate ligament reconstruction, including a body 20, and a connection member 30 and a pivoting member 40 disposed at the front end and the rear end of the body 20 respectively. The graft attachment device can cooperate with a connecting rod 50 and a handle 60 when operating.

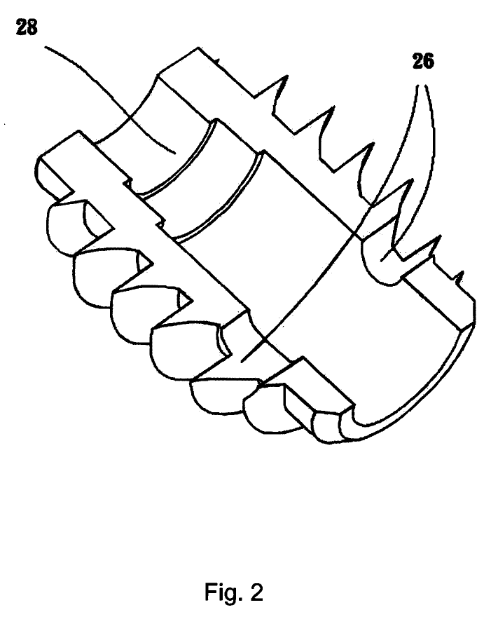

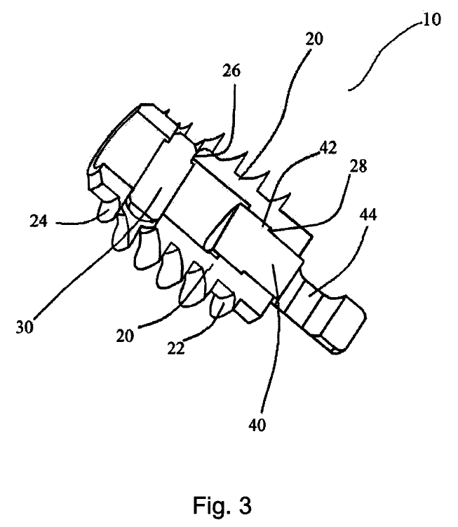

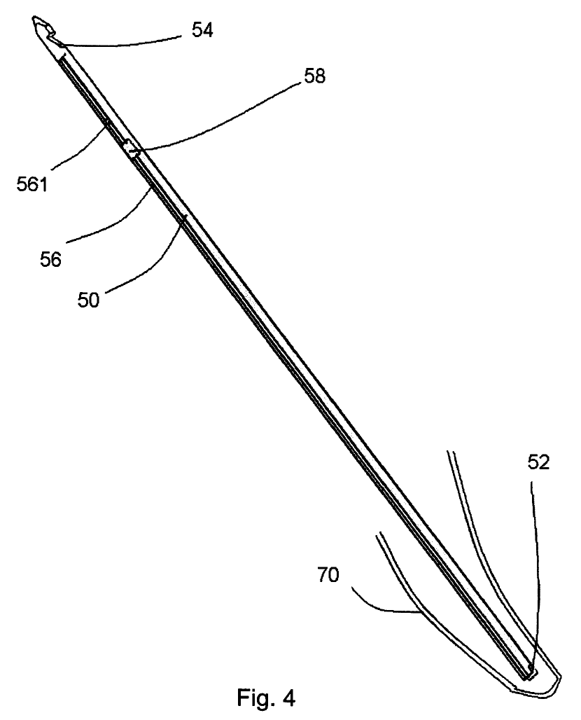

The body 20 with predetermined length is hollow, shown as FIGS. 2 and 3, and has a screw portion 22 formed at the outer edge of the body 20 including a threading area 24 corresponding to the front end of the body 20 for screwing on a bone when the body 20 moves forward and rotates axially, and two fixing portions 26 with through hole shape disposed adjacent to the front end of the body 20 respectively and spaced apart 180 degree and penetrated the inner edge and the outer edge of the body 20. The inner edge of the hollow rear end of the body 20 has a large diameter portion disposed adjacent to the distal end of the inner edge of the hollow rear end of the body 20, a small diameter portion disposed adjacent to the inside of the inner edge of the hollow rear end of the body 20, and a shoulder portion bridged therebetween.

The body 20 formed a hollow shape is for the consideration of preventing from over weight and volume. For the combination with other elements and operation method described next, it is also available for two blind holes disposed at the front end and the rear end of the body 20 to accommodate the connection member 30 and pivoting member 40 therein respectively.

The connection member 30, referring to FIGS. 2 and 3, is rod-shaped and two ends of the connection member 30 is disposed at the front end of the body 20 and embedded in the corresponding two fixing portions 26 respectively. Since the outer diameter of the connection member 30 is smaller than the inner diameter of the hollow front end of the body 20, a gap is formed between the outer edge of the connection member 30 and the inner wall of the body 20 for providing a rope-type member to pass through.

The pivoting member 40, referring to FIGS. 2 and 3, is rod with predetermined length. A stop portion 42 with a larger outer diameter is disposed at the front end of the pivoting member 40 which is stopped by the shoulder portion 28 to make the pivoting member 40 freely rotate axially but not to break away out of the body 20 after the stop portion 42 extends to inside the rear end of the body 20. A long-shaped through hole 44 is disposed at the rear end of the pivoting member 40 for installing a ligament thereon.

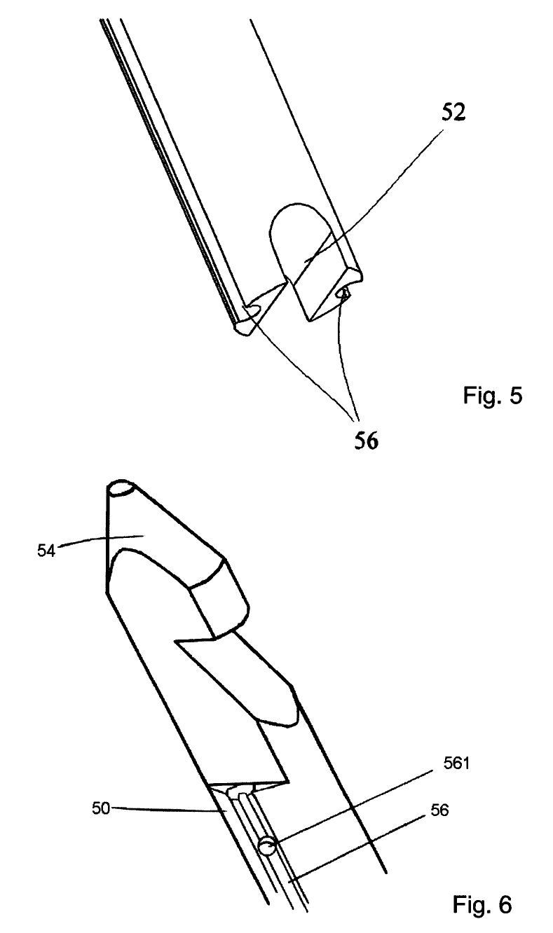

The diameter of the connecting rod 50 is smaller than the outer diameter of the body 20. The connecting rod 50 of this embodiment is inserted in the body 20. A pair of flanges are extended axially and disposed at one end of the connecting rod 50, shown as FIG. 5, and a cutout 52 is defined between the two flanges and bridged on the outer edge of the connection member 30. A clamped portion 54 is formed at the other end of the connecting rod 50 with flat hook shape, shown as FIG. 6. Two grooves 56 are disposed at the outer edge of the connecting rod 50 and extended axially and one end of each groove is extended out of the distal end of the two flanges respectively. A concave portion 58 is disposed at the outer edge of the connecting rod 50 and across one of the two grooves 56.

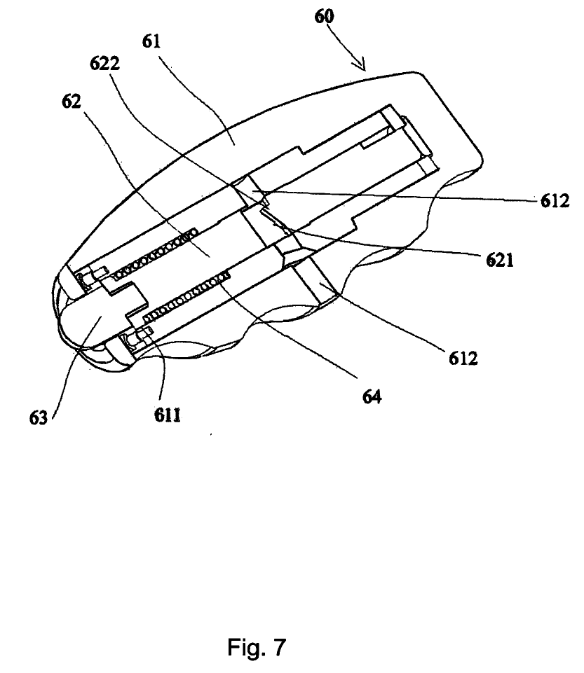

The handle 60, shown as FIG. 7, for easily connected with the clamped portion 54 or released is including a shell body 61, a first through hole 611 penetrated the shell body 61, a second through hole 612 perpendicular to the first through hole 611 and extended inwardly from the outer edge of the shell body 61 and pass through the first through hole 611, a sliding member 62 disposed and reciprocated in the first through hole 611, a press button 63 connected one end of the sliding member 62, and a spring 64 disposed in the shell body 61 and constantly forced the press button 63 outwardly to make the press button 63 slightly protrude out of the shell body 61 via the first through hole 611, wherein a third flat through hole 621 is disposed at the sliding portion 62 corresponding to the second through hole 612, and a hook 622 is pre-set in the third through hole 621 corresponding to the clamped portion 54.

The graft attachment device 10 must be combined with the connecting rod 50 first and a ligament 90 must be fixed in the long-shaped through 44 of the rear end of the pivoting member 44 in advance. The cutout 52 disposed at the distal end of the connecting rod 50 is bridged on the connection member 30, so that the connecting rod 30 is able to drive the body 20 to rotate with. And then, two ends of a rope-type member 70 (the steel wire is the best in this embodiment) are embedded at the two grooves respectively, and the distal end is fixed by a fix hole 561 pre-set on the connecting rod 50, wherein the middle portion pass through the gap between the connection member 30 and the body 20 and gone round the outer edge of the connection member 30, so that the connecting rod 30 is able to drive the body 20 to reciprocate with.

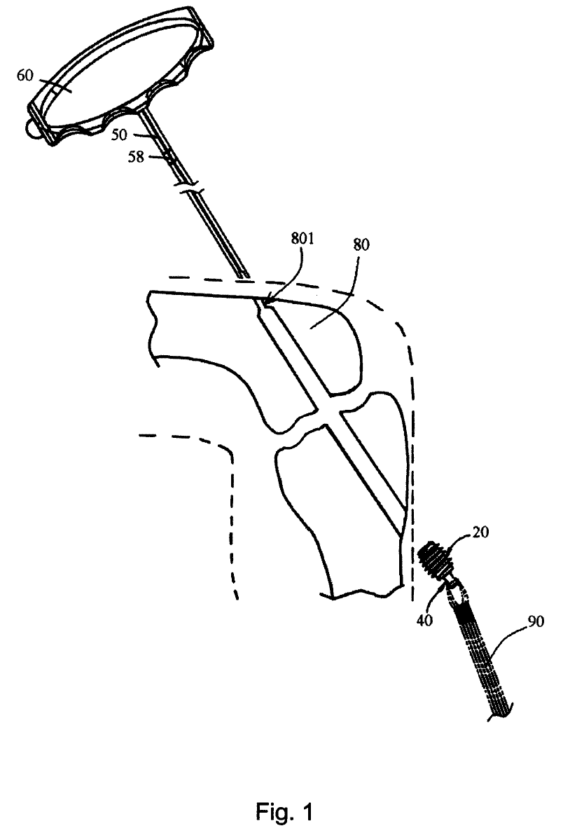

The operation method is boring a step hole from tibia to femur 80 in advance and making the inner diameter of the distal end 801 of the through disposed above the femur 80 smaller than the outer diameter of other portions and the body 20, shown as FIG. 1, and making the clamped portion 50 of the connecting rod 50 insert in tibia and out of femur with assembled body 20 and connecting rod 50 but the body 20 connected at the distal end be not able to pass through the distal end of the through hole 801. The operator is able to rotate the connecting rod 50 right away and screw therein by the threading area 24 disposed at the front end of the screw portion 22 when keeping inserting deeply in femur. After the depth which the body 20 is screwed in femur 80 is conformed to requirement by observation, the operator is able to cut the rope-type member 70 at the concave portion 58 by wire nippers, and then remove the connecting rod 50 and leave the graft attachment 10 in femur.

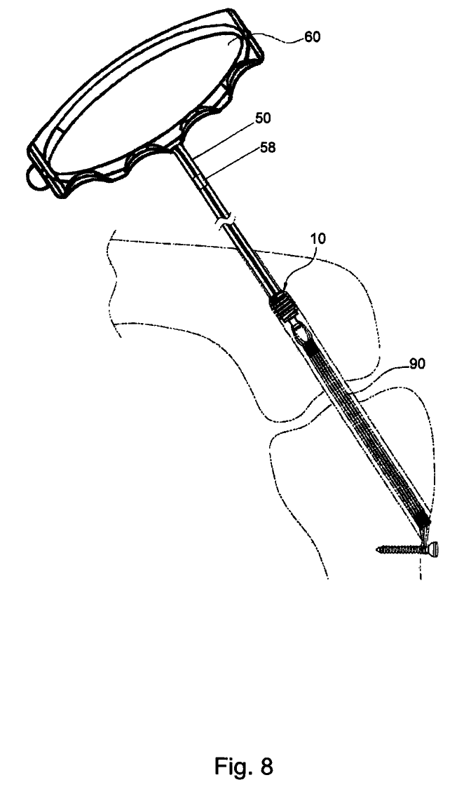

The handle 60 is for easily pulling and rotating the connecting rod 50 by operator, and the distal end (the portion is shown in FIG. 6) is inserted in the second through hole 612 and the third through hole 621 and locked by the hook 622 with hook shape after the clamped portion 54 pass out of femur. So that the operator pulls and rotates the connecting rod 50 by forcing outwardly and the femur 80 corresponding to the distal end 801 of the through hole is threaded and screwed therein by the graft attachment device 10. When the graft attachment device 10 is positioned, shown as FIG. 8, the operator is able to release the connecting rod 50 by pushing the press button 63.

By the above mentioned elements' cooperation of this invention, the connecting rod 50 for driving the graft attachment device 10 to thread and fix is connecting at the front end, and the ligament for accepting the construction is disposed at the rear end which do not interfere each other. Since the ligament 90 is freely pivoted at the rear end of the graft attachment device 10, the ligament 90 do not rotate with the connecting rod 50 to become twist in operation process, so as to prevent from damaging.

Although the invention has been explained in relation to its preferred embodiment, it is not used to limit the invention. It is to be understood that many other possible modifications and variations can be made by those skilled in the art without departing from the spirit and scope of the invention as hereinafter claimed.

What is claimed is:

1. A graft attachment device for cruciate ligament reconstruction, comprising:
a body, having a screw portion disposed at an outer edge of the body and the screw portion having a threading area corresponding to a front end of the body;
a connection member, fixed at the front end of the body, and a gap between the connection member and a corresponding portion of the body providing one rope-type member pass through; and
a pivoting member, having a front end rotatably pivoted at a rear end of the body, and a connection portion disposed at a rear end of the pivoting member for installing a ligament thereon.

2. A graft attachment device for cruciate ligament reconstruction as claimed in claim 1, wherein the body has a axial hole run through the front end of the body.

3. A graft attachment device for cruciate ligament reconstruction as claimed in claim 1, wherein the front end of the body is hollow, and the connection member is rod-shaped and two ends of the connection member bridge at an inner wall disposed at the front end of the body.

4. A graft attachment device for cruciate ligament reconstruction as claimed in claim 3, wherein the rear end of the body is hollow, the front end of the pivoting member pivots inside the rear end of the body, and the connection member exposes out of the rear end of the body.

5. A graft attachment device for cruciate ligament reconstruction as claimed in claim 4, wherein the body has a small diameter portion disposed inside the body adjacent to the hollow rear end, a large diameter portion disposed therein, and a first shoulder portion formed between the large diameter portion and the small diameter portion, and the pivoting member extends to a front end disposed therein and forms a corresponding stop portion.

6. A connecting rod for operating the graft attachment device for cruciate ligament reconstruction as claimed in claim 1, having a predetermined length, an outer diameter is smaller than an outer diameter of the screw portion of the body, and a driving portion combined to the connection member to drive the body and the connecting rod to rotate synchronously.

7. A connecting rod for operating the graft attachment device for cruciate ligament reconstruction as claimed in claim 6, wherein the driving portion is disposed in at least one cutout formed at a distal end of the connecting rod, and bridged at an outer edge of the rod-shaped connecting member.

8. A connecting rod for operating the graft attachment device for cruciate ligament reconstruction as claimed in claim 6, wherein a pair of grooves are formed outside the connecting rod extended along a long-axis direction of the connecting rod, and at least one end of the grooves is extended to the connecting rod for disposing in a distal end of the cutout.

9. A connecting rod for operating the graft attachment device for cruciate ligament reconstruction as claimed in claim 8, further comprises a rope-type member with adaptive flexibility having two ends disposed in corresponding grooves and a middle portion gone round a corresponding portion of the connection member for drawing the body to move along a long-axis direction of the body.

10. A connecting rod for operating the graft attachment device for cruciate ligament reconstruction as claimed in claim 6, wherein the end against the cutout forms a clamped portion for being clamped easily by an adaptive tool.

11. A connecting rod for operating the graft attachment device for cruciate ligament reconstruction as claimed in claim 8, wherein the end against the cutout forms a clamped portion for being clamped easily by an adaptive tool.
